(19) United States
(12) Patent Application Publication
(10) Pub. No.: US 2015/0216641 A1
(43) Pub. Date: Aug. 6, 2015

(54) MULTIFUNCTIONAL ORTODONTIC BRACKET COVER

(71) Applicants: Liviu Popa-Simil, Los Alamo, NM (US); David Ferl Jones, Los Alamos, NM (US)

(72) Inventors: Liviu Popa-Simil, Los Alamo, NM (US); David Ferl Jones, Los Alamos, NM (US)

(21) Appl. No.: 14/171,559

(22) Filed: Feb. 3, 2014

(51) Int. Cl. A61C 19/04; A61B 5/145; A61B 5/01; A61B 5/11; A61C 5/14; A61B 5/00

(52) U.S. Cl. CPC A61C 19/04; A61C 5/14; A61B 5/0017; A61B 5/0022; A61B 5/01; A61B 5/1111; A61B 5/14507; A61B 5/7405; A61B 5/7455

(57) ABSTRACT

A new method to measure and study in situ the chemical and mechanical stress of the orthodontic fixtures and to protect them by creating a feedback system for the patient that provides a warning on each exposure to an associated hazard. The device is an individual orthodontic protection cover applied over the arch wire and fixed on the orthodontic fixture by an elastic clamp and set over the tooth with a solid filler material to seal all the inner exposed surface of the tooth.

Inside the shell some electronics, powered by a micro-battery, is embedded to measure the pH, temperature, vibration, movement, force on the tooth, etc. using specialized transducers such as piezoelectric and pH transducers attached on the protective cover. The measured value is transmitted outside via radio frequency, or modulated light, or other signal protocol, to a specialized receiver that stores and analyzes it. The warning signals are transmitted to the patient via vibrations on the tooth, light, or other signaling method, asking for corrective actions such as tooth wash or the use of other less tooth aggressive food, establishing a feedback loop and educating the patient on dental hygiene and safety.

### CROSS REFERENCE TO RELATED APPLICATIONS

This application claims the benefit of U.S. Provisional application U.S. Ser. No. 14/942,394 and no International Application.

### BACKGROUND

Orthodontic treatment can improve mastication, speech and appearance, as well as overall health, comfort, and self-esteem. However, like many other interventions, orthodontic treatment has inherent risks and complications of which most important are, decalcification, enamel wear or fracture; pulpitis, gingivitis, burns, soft tissues direct trauma, mucosal ulceration due to appliances, trauma from headgear whisker, nickel-induced sensitivity associated with headgear, allergy/sensitivity to nickel, cytotoxicity, swallowing or aspiration of small parts and finally psychological.

Oral hygiene instruction is essential in all cases of orthodontic treatment, and the use of adjuncts such as sonic electric toothbrushes, interproximal brushes, chlorhexidine mouthwashes, fluoride mouthwashes, and regular professional cleaning must be reinforced. However, patient motivation and dexterity are paramount in the success of hygiene. Moreover, there will always be individuals whose oral hygiene is unsatisfactory. Experience shows that, in those who are unable to maintain a healthy oral environment, fixed orthodontics will fail spectacularly with braces in place due to the fact that the actual profile of the fixtures is complicated, with many corners, small holes and nooks, making it hard to reach by those skilled in the oral hygiene, and in children, who are inexperienced in oral hygiene, the risk of failure is significant.

Leaching of materials from appliances is responsible for hypersensitivity reactions and may entail the release of known allergens such as nickel, chromium, and cobalt. Other allergens are components or chemical catalysts in bonding materials, cold curing acrylics, or in latex components. However, there was no significant difference in nickel or iron concentrations between controls and subjects in whom the appliances had been in place for a number of weeks. The clinical significance of nickel release is as yet unclear, but should be considered in sensitive patients. A few patients have suffered severe latex allergies caused by elastics or operators' gloves. Allergy to nickel is more common in extraoral settings, usually as a result of contact with face-bow or headgear straps. Over 1% of patients have some form of contact dermatitis to zippers and buttons/studs on clothing, and about 3% of the latter claim to have experienced similar rashes with orthodontic appliances. Use of sticking plaster over the areas in contact with the skin is sufficient to relieve symptoms. Allergy to latex 39 and bonding materials have been reported, although these are rare.

Trauma such as lacerations to the gingivae and oral mucosa may present conditions such as ulceration or hyperplasia. They often occur during treatment or between sessions because of archwires, brackets and bands, especially where long unsupported stretches of wire rest against the lips. Excessive muscular activities of the cheek or tongue may act as triggers. The use of dental wax over the bracket may reduce trauma and discomfort, as may rubber tubing on the unsupported arch wire. Careful rounding-off of sharp edges of the appliance can also be helpful.

Extraoral appliances cause both extra- and intra-oral adverse reactions. Reports of injuries with extraoral appliances have shown that out of the nearly 5000 orthodontists (responsible for treating approximately 4.5 million patients), 4% reported that headgear injury had ensued in one or more of their patients; 40% were extraoral injuries. Samuels and Jones classified the types of injuries as follows:
accidental disengagement when playing;
incorrect handling;
disengagement by another child; and
disengagement while asleep.

There is a risk of damage and infection of the eye because the surface of the inner arch of the face-bow is rich in oral microorganisms and the eye forms an excellent culture medium following inoculation of microorganisms, even through a small abrasion. An infection of the eye is very hard to manage despite appropriate antibiotic therapy. These are a few of the incidents and conditions that this invention intends to prevent and minimize.

The U.S. Pat. No. 8,220,195 discloses a novel orthodontic bracket system including orthodontic appliances, such as brackets or buccal tubes, with a vertical instead of the conventional horizontal slot orientation. The occlusal-gingival slot orientation minimizes the risk of unintentional debonding of the bracket as a result of insertion and removal of the archwire. An archwire retention mechanism designed to guide, retain and/or seat the archwire. The archwire retention mechanism releases the archwire from the archwire slot whenever appropriate force is applied, or manipulation of physical properties of the material are altered by the operator.

Patent US 2005/0095549 A1 An orthodontic appliance includes a latch that comprises at least one clip with a region for receiving an archwire. The clip also includes a recess for receiving a post of the appliance, and the recess is not in communication with the archwire-receiving region. The post functions to secure the clip to remaining components of the appliance, such as a body or base of the appliance. Optionally, the clip may include a second recess for receiving a second post of the appliance.

U.S. Pat. No. 7,686,613 teaches about a ceramic orthodontic bracket has a centrally located clip for retaining an archwire in an archwire slot. The body of the bracket includes a mesial section, a distal section, and a bridge section, wherein the bridge section extends through, and retains, the clip. The bridge section also includes a recess adjacent to the bridge section that facilitates debonding of the bracket from a patient's tooth. Preferably there are gaps on one or both sides of the clip to allow space for debonding of the bracket by applying a mesial-distal compressive force on the bracket and fracturing the bracket along the recess. This configuration allows the overall mesial-distal width of the bracket to be reduced, thereby facilitating the bonding of brackets to narrow and/or maloccluded teeth. Another embodiment includes a stop member that is connected to the base of the bracket and retains the clip on the bridge section.

In the U.S. Pat. No. 7,377,777 is described an orthodontic appliance includes a latch that comprises at least one clip for retaining an archwire in an archwire slot of the appliance. The clip includes a first section, a second section opposed to the first section and a third section that interconnects the first section and the second section. The third section is adjacent the base and includes an outer edge having a convex shape in contact with a wall portion of the base. The convex shape facilitates opening the clip during insertion of an archwire into the archwire slot of the appliance.

Patent application U.S. Ser. No. 12/570,340 teaches about a self-ligating orthodontic bracket has an archwire slot, two outer tracks extending in an occlusal-gingival direction on the outer lateral surfaces of the bracket, and a vertical trough extending in an occlusal-gingival direction between the outer tracks. A clip has two parallel outer arms and a central tongue between the outer arms. The outer tracks of the bracket slidably engage the outer arms of the clip and the central tongue is slidably engaged by the vertical trough of the bracket, thereby allowing the clip to slidably move between an open position in which the outer arms of the clip are retracted from the archwire slot to allow an archwire to be placed into the archwire slot, and a closed position in which the outer arms of the clip extend across the archwire slot to retain the archwire in the archwire slot.

The patent application U.S. Ser. No. 10/730,344 presents an orthodontic appliance has a ceramic body and an archwire slot liner that is received in a channel of the body. The archwire slot liner has occlusal, gingival and lingual sections, and the lingual section is thicker than the occlusal or gingival section. The archwire slot liner helps resist fracture of the ceramic when the appliance is in use in the oral cavity, and yet need not adversely affect the aesthetic appearance of the appliance. A notch in the archwire slot liner is aligned with a channel in the body and provides a passageway for receiving a vertical auxiliary device.

The U.S. Pat. No. 7,988,711 develops a skeletal stabilization system, which include a base, a longitudinal member, and a retaining member. The base includes an engagement surface with a surface area that is positionable in contact with a bone to atraumatically or traumatically engages the base to the bone. The retaining member engages the base to retain the longitudinal member relative to the base so that the longitudinal member can provide a desired stabilization effect to one or more adjacent bony portions.

The U.S. Pat. No. 7,611,352 is about an orthodontic bracket system including a plurality of interchangeable ligation covers so as to allow the patient and practitioner to create a desired aesthetic look. The inventive orthodontic bracket system includes a bracket base, at least one arch wire slot formed in the bracket base adapted to receive an arch wire therein, a first interchangeable ligation cover that when releasably assembled to the bracket base can be selectively moved relative to the bracket base between an open non-ligating position relative to the arch wire slot and a closed, ligating position relative to the arch wire slot, and a second interchangeable ligation cover of a different color and/or formed of a different material relative to the first cover. The interchangeable ligation covers can be removed and replaced with another cover of a different color and/or different material to create a different aesthetic appearance.

The U.S. Pat. No. 7,963,767 talks about a self-ligating orthodontic brackets include a bracket base, at least one arch wire slot formed in the bracket base adapted to receive an arch wire therein, and a ligation cover which is selectively slidable relative to the bracket base between an open non-ligating position relative to the slot and a closed, ligating position relative to the slot. The exterior labial surface of both the base and ligation cover comprise a compound two axis curvature so that substantially all of the exterior labial surface of both the bracket base and the ligation cover present a smooth, continuous exterior surface that is substantially free of sharp discontinuities and crevices.

Teeth are protected by saliva. Saliva has a variety of different functions, including helping food go down—but it is very important for our teeth. Normally saliva is alkaline, with a pH of 7.2 or above. At this pH, the saliva encourages strengthening and the repair of teeth. However, the alkaline saliva in one's mouth may change and become acidic in a stressful situation (ever get that icky taste in your mouth?), or even with a simple health change such as a sinus infection. A way to find out the overall pH balance (or imbalance) of your body is to test the pH of your saliva. Saliva pH can range anywhere from 5.5 to 7.5 or more. If one has enough alkaline minerals, your saliva test should register a nice 7.0 to 7.5. But if the body's mineral reserves are too low, the acids will overwhelm your system, and you'll test under7.0.

The normal ph level of a mouth should be 6.5 or higher. Certain foods such as potatoes, milk, and bananas will raise a mouth's ph level. Water tends to vary in pH and effects the mouth as such. Hormonal imbalance, dry mouth, and gastric acids can all contribute to making a mouth more acidic. After consuming acidic food or drink, it typically takes about 20 minutes for a mouth to return to the correct ph level.

Some people even test as low as 4.5-5.75, which is not where one wants the pH to be. In fact, if pH tests that low, we recommend one take immediate action to correct this acidity. Because the pH scale works like the Richter scale—i.e. logarithmically—a pH of 5.0 is 100 times too acidic! The recommend pH level for the mouth is between 6.4-7.2.

The pH of the mouth will help determine which bacteria (good or bad) are able to survive and receive nutrients. Low pH (acidic) saliva supports bacteria that are more harmful to your teeth and more alkaline (neutral) saliva supports bacteria less harmful to your teeth.

Low pH in mouth (acidic) stimulates a breeding ground for Bad Bacteria, and increased susceptibility of Cavities and Gum Disease

### SUMMARY

The present invention relates to an orthodontic bracket cover made with smooth surfaces and sheathed with materials compatible with the mouth in order to prevent lingual or palatal side swallowing and itchiness inside the mouth of orthodontic patients.

Bracket covers of the present invention, are set on the orthodontic wire, and are also held in place by the shape of the bracket. The orthodontic appliance comprises a round and smooth body shell that covers the bracket plate that is bonded to a tooth and extends to touch the tooth surface on all sides. It is tightened to the bracket wire and bracket body with elastic clamps and has the space surrounding the bracket down to the tooth filled with compatible rubber or other compatible filler material to prevent the accumulation of food and other chewing byproducts. The cover can be removed from the bracket by simple operations since it is fixed and connected thereto with a connecting part and elastic connecting device. The cover inner space contains a micro light emitting diode (LED) device, a battery, and a ph sensor that, when the mouth becomes acid or too basic, starts flashing, making the patient aware of the need of a tooth brush or mouth wash.

The arch wire emerges from the cap through two rounded slits, each having an internal elastic fixture that secures the cover to the wire. A stiff rubber or other compatible material is also poured and solidified inside, taking the shape of but without sticking to the tooth or the bracket, in order to be easy removed by the orthodontist.

In order to protect the tooth from too much physical force, a microelectronic device may be added inside the cover to sense the pressure on the cover and activate a piezo-mini-actuator to act like a buzzer and let the patient know that excessive force was applied in the tooth. It also activates the LED or applies an electric signal that can be directly generated by the piezo-electric material and can be felt in the gum of the patient. This device will protect the orthodontic implant against involuntary excessive force.

A temperature micro-sensor may also be added to detect the temperature differences and warn the user by illuminating the LED or buzzing inside the cover, developing a feedback loop, meant to protect the tooth bracket and the operation.

The dangerous pH may also be sensed as a voltage difference between the arch wire and the metal of the bracket cover, thus giving an indication if it is too basic or too acid in order to trigger the patient to use mouth wash.

### BRIEF DESCRIPTION OF THE DRAWINGS

FIG. 1—Schematic view of three teeth having covered brackets

FIG. 2—Front view of two teeth having covered orthodontic brackets

FIG. 3A—Cross section through an orthodontic covered bracket

FIG. 4—Longitudinal section through an orthodontic covered bracket

FIG. 5—Front view of an orthodontic bracket cover

### DETAILED DESCRIPTION OF THE INVENTION

The inventors consider that most of the problems generated by the actual orthodontic operations are due to changes in the shape of the face, jaw and cheekbones that may occur as a result of braces. The shape of the actual braces, in spite of optimization, favors plaque buildup when food is retained in and around braces. It is important but difficult to maintain proper oral hygiene by brushing and flossing thoroughly when wearing braces to prevent tooth decay, decalcification, or unpleasant color changes to the teeth.

The presence of various metals such as stainless steel, Ni, Ti, etc., in alloys may trigger a small chance of allergic reaction to the metal or to the elastics used in braces. In even rarer cases, latex allergy may result in anaphylaxis. Latex-free elastics and alternative metals can be used instead if it is previously known that this reaction might occur in a particular patient.

The most common effect is mouth sores that may be triggered by irritation from components of the braces. Many products can increase comfort, including oral rinses, dental wax or dental silicone, and products to help heal sores, but their sharp shape favors electro-pile effect and pressure effect that wounds the inner part of the cheek. Pain and discomfort are common after adjustment and may cause difficulty eating for a time, often a couple of days.

Braces can also be damaged if proper care is not taken. It is important to wear a mouth guard to prevent breakage and/or mouth injury when playing sports. Certain sticky or hard foods such as taffy, raw carrots, and hard pretzels should be avoided because they can damage braces. Frequent damage to braces can prolong treatment. Some orthodontists recommend sugar-free chewing gum in the belief that it may expedite treatment and relieve soreness; other orthodontists object to gum chewing because it is sticky and may therefore damage the braces.

The metallic look may not be desirable to some people, although transparent varieties are available. According to a survey published in the American Journal of Orthodontics and Dentofacial Orthopedics, dental braces with no visible metal were considered the most attractive. Ceramic braces with thin metal or clear wires were a less desirable option, and braces with metal brackets and metal wires were rated as the least aesthetic combination.

The devices described here represent the means to apply this novel method via a family of covers with various dimensions and materials that may accommodate all the patients preferences and at the same time reduces the electro-pile effect inside the mouth. The smooth shape reduces the effort in the pads and the hooks and nooks where the food may be grabbed and retained. The active part of the system is elastic shaped memory wire that penetrates the covers.

The microelectronics inside the cover may perform various functions, of which the most important is the pH detection and alert. When the mouth acidity passes the normal range, the pH sensor simply detects that and lets the patient know that and that he/she needs a mouthwash or rinse in order to reset the mouth's normal chemistry. Other functions such as stress detection and temperature detection may also be helpful, aiding with education and habits, particularly with the youth.

### 2. Best Mode of the Invention

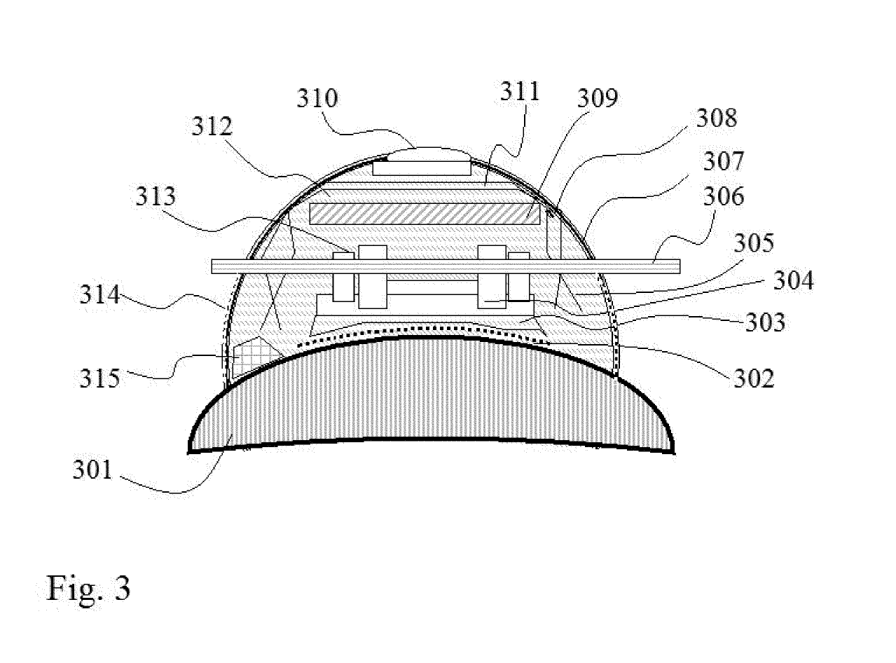

FIG. 3 shows the best mode contemplated by the inventors of the protection cover sheath of an orthodontic bracket. The cover shell is attached over the bracket using a removable fixture made of elastic wire in the form of a clamp that holds the bracket on and may be easily removed. Inside there is a micro-battery powering microelectronics.

There are some holes into the shell for the pH sensors and the led to pass through. The piezoelectric transducer is used to measure vibrations but also to apply vibrations to the tooth to warn the patient of some situations that triggered the electronics. A radio frequency communicator is used to send the measurements outside the mouth to a reception device, such as a blue-tooth capability, that logs the data and provides them for analysis.

The simplest application consists in the usage of the profiled cover sheath made of a mouth compatible material to be applied over the orthodontic bracket in order to prevent the newly set brackets from hurting the skin and make wounds. The cover is fixed on the arch wire and is filled inside with a elasto-plastic or other compatible material that also holds it on the bracket's surface without sticking, but sealing all the tooth surface and preventing moisture and food from being stuck underneath.

### 3. How to Make the Invention

As can be amply seen from the drawings the bracket cover may be made as an earring having a spherical or ellipsoidal shape, empty inside, because at first approximation it may be manufactured from gold. It may also be manufactured from ceramic, resins, plastic or other alloys, that are compatible with the mouth environment, especially with the nearby cheek's inner skin. The fixtures are used to hook on arch wire. Two slits are made in order to allow the arch wire to pass through and be easy removed. It may be made from any metal, even aluminum or tin alloy if properly coated and painted with appropriate coating materials. In order to be stiff on the bracket the inner space is filled with a high density rubber or other compatible material that accommodates the shape and position of the bracket and the borders and seals to prevent water and saliva carrying food residue that can fermentation damaging the tooth.

Inside the inner space there is enough room for microelectronics devices that may provide useful functions. One of these is to measure the temperature and signal if it is too hot or too cold. To do that one has to insert a micro-battery in the room between the bracket and the cover, along with the necessary microelectronics. A temperature sensor and a differential amplifier would be added. There are several ways to make the user aware of the extremes, by making a led light, by installing a buzzer on the tooth and by an electric discharge. The preferred methods are the light and vibration. To see the light from the LED it would have to protrude through the sheathing, and for vibration a transducer would have to be installed on the tooth's surface.

Another very important measurement is that of pH, because it determines the corrosion speed of the tooth and the bacterial growth. To measure pH one may use the difference in electrochemical potential between the sheathing and the arch wire, or may install electrodes through holes made in the protective case, using metals compatible with the mouth and thereby preventing a change in taste. It is there possible to measure the food-tooth pH or the tooth-gum pH, or both.

These measurements are not enough, it is important to also create a feedback loop that allows the patient to immediately bring the mouth to the right chemistry and/or to be transmitted outside to a data logger, using blue tooth, modulated light (either IR or visible) and the appropriate detector.

The force, vibration measurement is also important and may be performed accurately using piezoelectric transducers. The device may now be used alone, without braces, just using a single bracket pad to stick on the tooth and with the electronics, for research purposes, for treatment of symptoms affecting the dental health, or for education, helping patients develop complex reflexes, and remember them long after the sensors have been removed.

### DETAILED DESCRIPTION

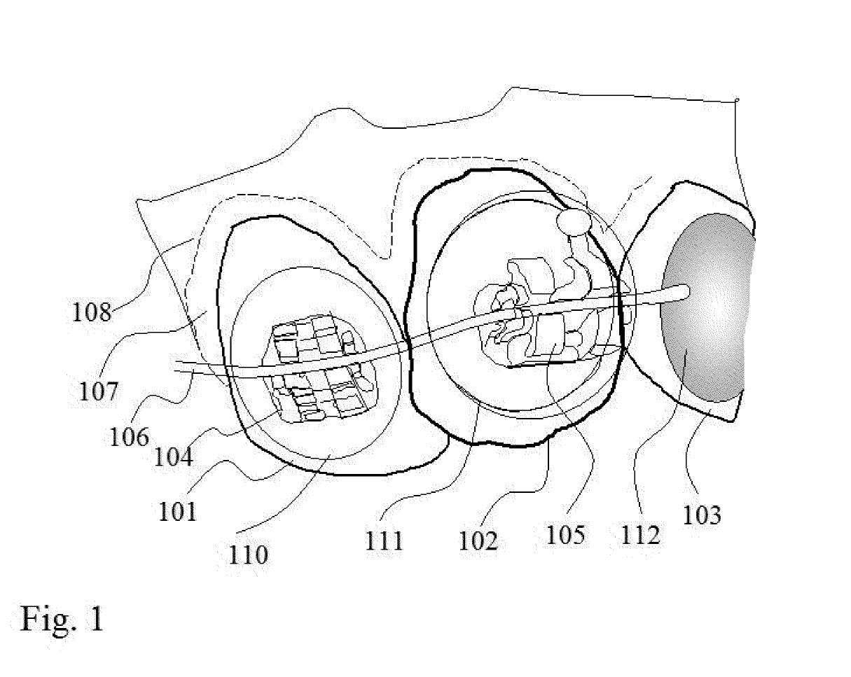

FIG. 1 shows a schematic view of three teeth having covered brackets. The tooth 101 is in a displaced position and has the bracket 104 glued on it, and so the tooth 102 nearby having the bracket 105 with a special bracket hook on it and the tooth 103 nearby that has the opaque cover 112. All the teeth have a gum 107 covering the teeth and the maxillary structure 108. In order to align the tooth in a pleasant mode a special arch wire 106 is used that is connected tight in all the brackets. The wire passes through the profiled covers 110, 111 and 112, using special slits. The cover is tight on the wire and on the bracket's body.

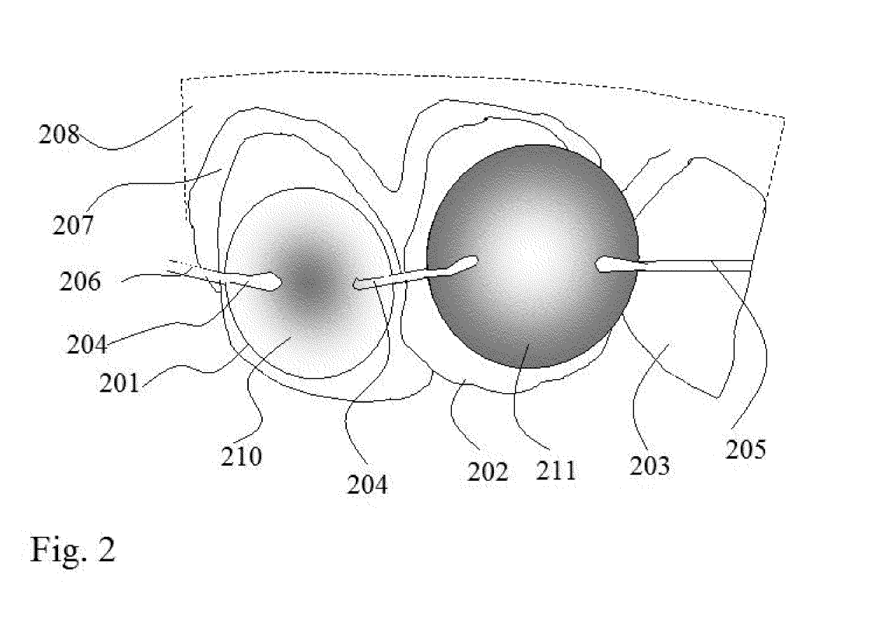

FIG. 2 shows a front view of two teeth covered with orthodontic brackets. The upper maxilary 208 has the gums 207 covering the teeth 201, 202 and 203 that is partially seen. The elastic wire 206 is connecting the tooth and is gently pushing to bring them to the right alignment. On the other side the wire 205 is going further without having a connection with the tooth underneath. The wire passes through the smooth shaped covers 210 and 211 through special cut slits 204 that have rounded smooth edges.

FIG. 3 shows a cross section through an orthodontic covered bracket placed on a tooth 301. The bracket's pad 303 is placed on the surface of the tooth using glue 302. The bracket has structure for arch wire 306 guidance 304. The cover 307 is fixed on the wire using elastic wire 305. The wires are welded or pressed in two buttons 308 on the case 307. An LED (Light Emitting Diode) 310 is placed on the outer shell and is controlled by the electronics 311 which is powered by the battery 309. The space inside 312 is fulfilled with solid silicone rubber or other compatible material leaving no room for liquids or residual food inside. The orthodontic bracket 304 may also contain supplementary hooks or anchors 313 inside the cover. On the lateral sides of the cover, that may look like a sphere or ellipsoid, slots 314 are cut to pass and accommodate the arch wire. The electronics inside may detect stress, temperature and/or pH and trigger a light warning signal or a sound-vibration buzzer that is felt by the patient so that he/she can take corrective action in order to maintain the teeth within safe parameters.

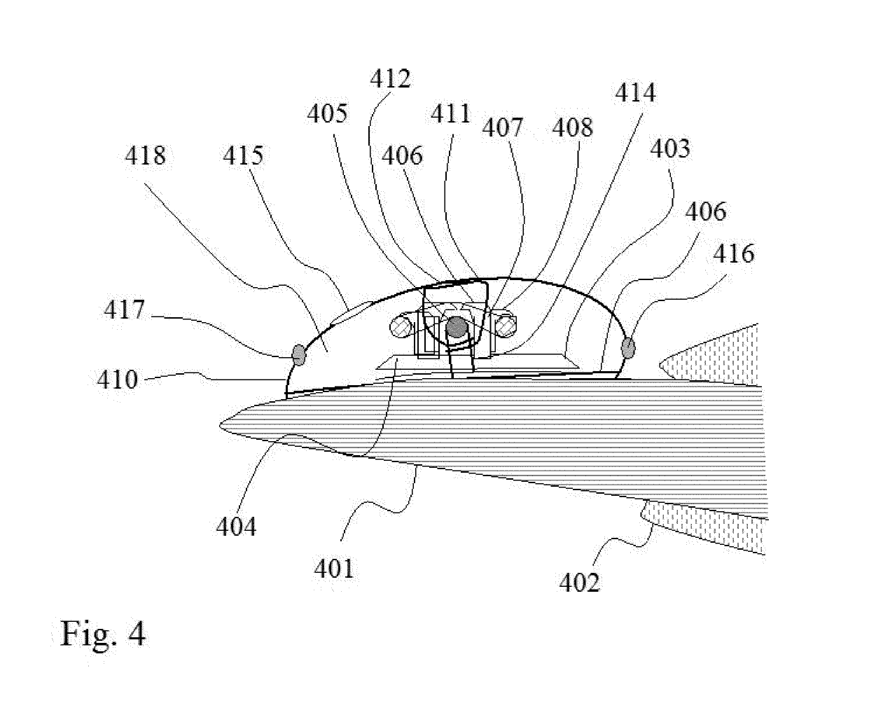

FIG. 4 shows a longitudinal section through an orthodontic covered bracket placed on a tooth 401 having a gum 402. The orthodontic pad 403 is glued on the tooth's 401 surface. The bracket 404 has a specific structure to guide and hold the arch wire 405, that is made of hooks 406 to hold the wire 405 and elastic ligature 407 that is stretched between the outer hooks 408 specially designed for this application. The cover 410 is connected tightly to the bracket 403 by using the arch-wire 405 which it hooks using steel clamps 411, that are attached to the shell by fixtures 412. The case 410 has two slits 414, rounded and smooth, which allow the arch wire 405 to be set into place.

The case contains a gum-tooth pH sensor 416 and a food tooth ph sensor 417, that are used to measure the alkalinity or acidity of the food and mouth saliva and if out of safety range triggers the warning procedure, making the led 415 pulse. Inside the small shell are microelectronics and filler 418 that does not stick on the bracket but fills its contours. This filler makes a safe, dry environment under the cover preventing the food or saliva from coming underneath the cover.

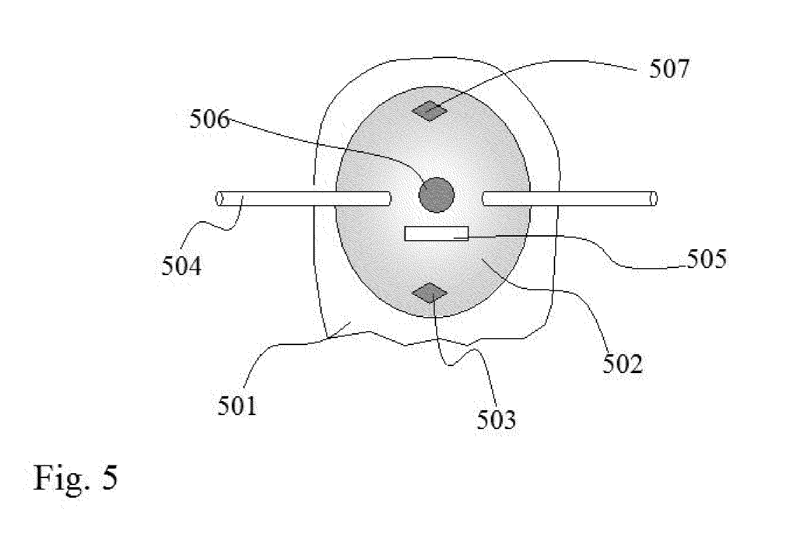

FIG. 5 shows a front exterior view of an orthodontic bracket cover. The tooth 501 has the bracket covered by cover sheathing 502, which attaches to the arch wire 504. The cover has an embedded food pH sensor 503 and a gum saliva pH sensor 507. The electronics beneath measures the signal and uses the LED 506 to warn of abnormal values and signal the need for urgent care. It may also use an electrode 505 placed on the surface of the cover which, when referenced to the shell or arch wire 504, can emit a signal (blue-tooth, 802.11 wi-fi, or other protocol) to be collected by cell phone apps or other data logging devices.

The usage of such a cover, without the orthodontic braces may be also done for medical or behavioral research purposes by using some covers equipped with sensor devices to measure the pressure on the tooth, number of chewing movements, eating habits, mouth chemistry and more, as a function of time, helping the specialists improve the dietary regime and detect chronic afflictions such as tooth grinding habits that drives to dental premature damage. It may also be used in education, by collecting and demonstrating conditions in the mouth.

The system as described above, when applied to kids, may be used in their early education, making them aware when the mouth environment is in danger by sending a alarm signal (vibration, light, or other) and thereby sending the kid to wash the his/her mouth or stopping a dangerous chewing situation. After wearing the system for a period of time, the patient will develop supplementary skills and extra sensorial abilities that will make the patient know and, by reflex, apply the correction to the mouth state without needing to wear the smart cover. That will be an accelerating factor in early dental education.

### BRIEF DESCRIPTIONS OF INVENTION

The present invention refers to a set of improvements to normal orthodontic brackets, providing them with a smooth compact cover that has multiple functions mainly designed to reduce the discomfort and wounds to the skin and to continuously alert the patient abnormal mouth conditions, preventing tooth degradation and educating the patient.

The main embodiment of the invention refers to the enhancement of the orthodontic bracket by adding the cover over the bracket, which provides a smooth flat surface where the inner side of the cheeks may be pressed against the fixture, thereby minimizing wound hazard, and eliminating the sharp angle surfaces where the food is easily retained and its fermentation drives to rapid tooth deterioration.

Starting from these needed developments a novel system of tools needed to assure enhanced performance and minimal harm to the body have been developed.

The new procedure will start with detection of the mouth alkalinity or acidity by a set of pH sensors driving the signal to a threshold circuit that activates a pulsed LED light or other signaling device, making the patient aware of the hazard and allowing the patient to apply corrective measures, most usually washing the mouth.

In the simplest form of application the invention is just a cover over the brackets made from a material compatible with the mouth tissue, as gaudent, gold, zirconia, stainless steels, etc. that is smooth enough not to hurt surrounding tissue and sealed to the bracket and wire.

### Examples of the Invention

Thus it will be appreciated by those skilled in the art that the present invention is not restricted to the particular preferred embodiments described with reference to the drawings, and that variations can be made therein without departing from the scope of the present invention as defined in the appended claims thereof. The present invention consists of the development of a set of improved orthodontic covers that may prevent brace mounting complications.

The application of these customized versions will extend the range of usage minimizing the negative impact of the treatment on the patient, and also reducing undesirable collateral effects and medical complications. The use of the embedded sensors will provide medical progress, providing the patient continuous mouth and tooth information such as pH, force on the tooth, temperature and thereby preventing the bad things that can happen to the teeth by announcing to the patient (by light, vibration, or other signal) that some corrective action is needed. Some derivatives of this equipment, without the function to protect orthodontic braces, may be used on people just to continuously control their mouth acidity, temperature differences, and biting force, and/or other conditions of feedback and after a period of time wearing the cover on a tooth and becoming aware of these conditions, allowing them to make appropriate corrections, thereby reducing the number of dental interventions. The application of teaching with this new feedback will generate a step forward in medicine, by intensively using multi-parameter monitoring and more body friendly invasive devices to further generate data about habits and stress applied to teeth, to better guide both the medical community and patients.

What is claimed is:

1. A tooth cover sheathing/shell comprising:
a. An external shell
b. An internal elastic and shaped fixture to secure it to the orthodontic bracket
c. A microelectronic system inside made of:
i. electrodes for PH measurement
ii. optical and vibration signaling devices
iii. battery
iv. specialized electronics
v. sensors and transducers
vi. wi-fi or optic communication devices
d. a set of thin protective shell covers
e. a space filler and sealant

2. A simple orthodontic bracket cover comprising:
a. A rigid smooth shaped rigid shell
b. Slits for adjusting the arch wire
c. Means of being attached on the arch wire
d. Space filler

3. A method of making tooth measurement and creating a feedback loop for protecting the orthodontic work and educating the patient using the tooth cover based on:
a. A protective cover shell attached on the tooth
b. Specialized measurement microelectronics
c. Sensors and transducers for measuring:
i. The tooth pH
ii. The tooth's temperature
iii. The tooth's force
iv. Tooth acceleration or vibration
d. Transducers for local signaling of warning levels to the patient by using:
i. Light pulses
ii. Sound and/or vibration
e. Communication systems using:
i. Modulated visible light
ii. Modulated IR light
iii. Radio frequency (in 802.11 or blue-tooth)
f. Systems of reception and data analyses

4. A tooth cover sheathing/shell according to claim 1 that uses a set of clamps to hold on the arch wire

5. A tooth cover sheathing/shell according to claim 1 that uses a set of electrodes that penetrates the shell to measure pH

6. A tooth cover sheathing/shell according to claim 1 that uses a led to signal the warning messages to the patient.

7. A tooth cover sheathing/shell according to claim 1 that uses a piezoelectric transducer to measure vibrations and stress to the case and to transmit a sound and/or vibration to warn the patient of a message.

8. A tooth cover sheathing/shell according to claim 1 that uses a chargeable battery or storage device to power the electronics.

9. A tooth cover sheathing/shell according to claim 1 that uses a modulated transmitter system using either or both IR or RF signals.

10. A simple orthodontic bracket cover according to claim 2 where the coversheet is made of a patient skin compatible material.

11. A simple orthodontic bracket cover according to claim 2 that may be profiled as an ellipsoid, with lateral slits for the arch wire.

12. A simple orthodontic bracket cover according to claim 2 that uses a plastic mold material to fill in the spaces and seal the shell on the tooth.

13. A simple orthodontic bracket cover according to claim 2 that can be painted or otherwise covered and shaped in various patterns and profiles.

14. A simple orthodontic bracket cover according to claim 2 using ceramic materials in the tooth's color to be a stealth on the tooth.

15. A method of making tooth measurements and creating a feedback loop for protecting the orthodontic work according to claim 3 where a microelectronics system is used to measure pH between the tooth and mouth or tooth and food.

16. A method of making tooth measurements and creating a feedback loop for protecting the orthodontic work according to claim 3 using direct feedback to the patient, that receives a light or vibration signal when a hazardous exposure appears on the tooth.

17. A method of making tooth measurements and creating a feedback loop for protecting the orthodontic work according to claim 3 based on measurement of the signal and transmitting them to an outside receiver using radio-frequency or IR/visible modulation.

18. A method of making tooth measurements and creating a feedback loop for protecting the orthodontic work according to claim 3 using piezoelectric transducers to measure the stress in tooth, its vibrations and to transmit signals to the patient.

19. A method of making tooth measurements and creating a feedback loop for protecting the orthodontic work according to claim 3 using the protection conductive sheeting and the arch wire to create an antenna to emit signals and a pH sensor

20. A method of making tooth measurements and creating a feedback loop for protecting the orthodontic work according to claim 3 using the system for oral hygiene education tutoring, and for studying the alimentary habits.
